(19) United States
(12) Patent Application Publication
(10) Pub. No.: US 2020/0207844 A1
(43) Pub. Date: Jul. 2, 2020

(54) ANTI-VEGF ANTIBODY

(71) Applicant: IGC BIO, INC., Cambridge, MA (US)

(72) Inventors: Dror BARAN, Tel Aviv (IL); Lior ZIMMERMAN, Tel Aviv (IL)

(73) Assignee: IGC BIO, INC., Cambridge, MA (US)

(21) Appl. No.: 16/646,093

(22) PCT Filed: Sep. 11, 2018
(86) PCT No.: PCT/US2018/050430

§ 371 Date: Mar. 10, 2020

Related U.S. Application Data
(60) Provisional application No. 62/557,155, filed on Sep. 12, 2017.

(51) Int. Cl. C07K 16/22; G01N 33/574

(52) U.S. Cl. CPC C07K 16/22; G01N 33/57492; G01N 2333/71; C07K 2317/622; C07K 2317/565

(57) ABSTRACT

This disclosure relates to antibodies and antigen-binding fragments that bind specifically to Vascular endothelial growth factor (VEGF), and prophylactic, diagnostic, and therapeutic methods of using the same.

### CROSS-REFERENCE TO RELATED APPLICATIONS

This application claims priority of U.S. Provisional Application No. 62/557,155, filed Sep. 12, 2017, which is hereby incorporated by reference.

### FIELD OF THE INVENTION

The invention relates to antibodies or antigen-binding fragments that bind specifically to Vascular endothelial growth factor (VEGF), and prophylactic, diagnostic, and therapeutic methods of using the same.

### BACKGROUND OF THE INVENTION

Monoclonal antibodies have been functioning as therapeutic, diagnostic and research agents since the 1970s. By 1986 the first therapeutic monoclonal antibody was approved for prevention of kidney transplant rejection and nowadays varied antibody fragments have become the biggest class in the biopharmaceutical market.

Current antibody candidates for biopharmaceutical development were originated by amplifying human B cells or synthesizing a completely artificial library. Antibodies cloned from B cells may not represent the full diversity of the immune system and also may have a bias towards certain clone sequences. Synthetic libraries may produce immunogenic antibodies that can potentially trigger an immune response in patients.

Therapeutic antibodies must fulfill a high standard with regard to their developability, stability, immunogenicity, and functional activity.

Most importantly, for an antibody to function as a drug, it may be required to inhibit, activate or block a cognate interaction between its target and a certain target interaction partner. For this effect to occur, the antibody has to bind the target at the same epitope as the interacting partner and with better (or no worse) affinity.

Angiogenesis is a tightly regulated process responsible for the development of new blood vessels from a pre-existing vascular network. During development and normal physiological processes such as wound healing and the menstrual cycle, angiogenesis is regulated by endogenous activators and inhibitors. A key step in tumor development, the ‘angiogenic switch’ occurs when endogenous activators of angiogenesis outweigh endogenous inhibitors, thereby shifting the balance of angiogenic mediators and stimulating angiogenesis. This results in increased blood vessel formation, which supplies growing tumors with necessary oxygen and nutrients for sustained growth, however the resulting vasculature is disorganized and poorly structured, leading to chaotic blood flow and leaky blood vessels. In absence of a blood vascular network, tumors are restrained in size due to limits in the diffusion of oxygen. Tumor angiogenesis is a hallmark of cancer, and is required for continued growth and metastasis. VEGF is a major mediator of angiogenesis in normal physiology and in cancer. There is an up-regulation of VEGF family members and the VEGF receptors in many different tumors, providing a target for cancer therapy. There are five members of the human VEGF family: VEGF-A VEGF-B, VEGF-C, VEGF-D and placental growth factor (PlGF), however it is VEGF-A (referred to hereinbelow as VEGF), that has been implicated in tumor angiogenesis.

This disclosure presents sequences of identified antibody fragments (scFVs) that bind Vascular endothelial growth factor (VEGF).

### SUMMARY OF THE INVENTION

In one aspect, the invention provides isolated antibodies or antigen-binding fragments, wherein said antibody or antigen-binding fragment is specific for human and murine Vascular endothelial growth factor (VEGF).

In another aspect, the invention provides methods of treating a tumor in a subject comprising the step of contacting the tumor with an antibody or antigen-binding fragment thereof described herein that is operably linked to a biologically active agent, wherein the agent is a toxin, a radioisotope, a nanoparticle or a bio-active peptide.

In another aspect, the invention provides methods of inhibiting angiogenesis in a solid tumor in a subject, the method comprising the step of contacting the solid tumor with an antibody or antigen-binding fragment thereof described herein that is operably linked to a biologically active agent, wherein the agent is a toxin, a radioisotope, a nanoparticle or a bioactive peptide.

In another aspect, the invention provides methods of inhibiting or suppressing a tumor in a subject, comprising the step of administering an effective amount of an antibody or antigen-binding fragment thereof described herein, wherein the antibody or antigen-binding fragment is operably linked to a biologically active agent, wherein the agent is a toxin, a radioisotope, a nanoparticle or a bio-active peptide.

In another aspect, the invention provides methods of delaying progression of a solid tumor in a subject, the method comprising administering to the subject an effective amount of an antibody or antigen-binding fragment thereof described herein, wherein the antibody or antigen-binding fragment is operably linked to a biologically active agent, wherein the agent is a toxin, a radioisotope, a nanoparticle or a bio-active peptide.

In another aspect, the invention provides methods of diagnosing the presence of a tumor or a cancer growth in a subject, the method comprising sampling a tissue sample isolated from the subject with a composition comprising an antibody or antigen-binding fragment thereof described herein, wherein specific binding of the antibody or antigen-binding fragment to the tissue sample is indicative of the presence of a tumor or cancer growth in the subject.

In another aspect, the invention provides methods of imaging a VEGF-containing tumor, the method comprising the step of applying an antibody or antigen-binding fragment thereof described herein that is operably linked to a secondary reagent; wherein the secondary reagent can be visualized once the antibody or antigen-binding fragment has bound its target VEGF.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1: Flow cytometry analysis of dual labeled yeast-display scFv of interest and its bound antigen Vascular endothelial growth factor (VEGF). Yeast-display scFv expression were detected by anti-c-myc mAb chemically labeled to AlexaFluor488 (sc-40 AF488, Santa Cruz Biotechnology) and bound antigen Vascular endothelial growth factor (VEGF) (VE5-H8210, ACROBiosystems) detection by AlloPhycocyanin (APC) Streptavidin (016-130-084, Jackson ImmunoResearch laboratories).

FIG. 2: Amino acid sequences of anti-VEGF scFv. The heavy chains are italicized and the light chains are underlined. The complementarity determining regions (CDRs) are shown in bold.

FIG. 3: Amino acid sequences of the VEGF ligand used in the study.

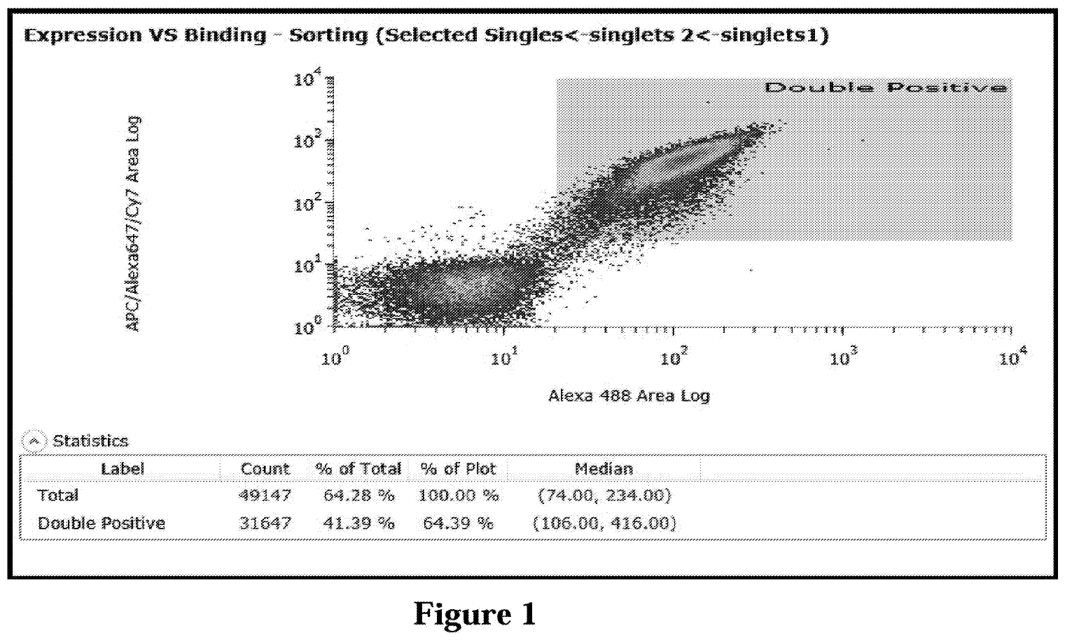

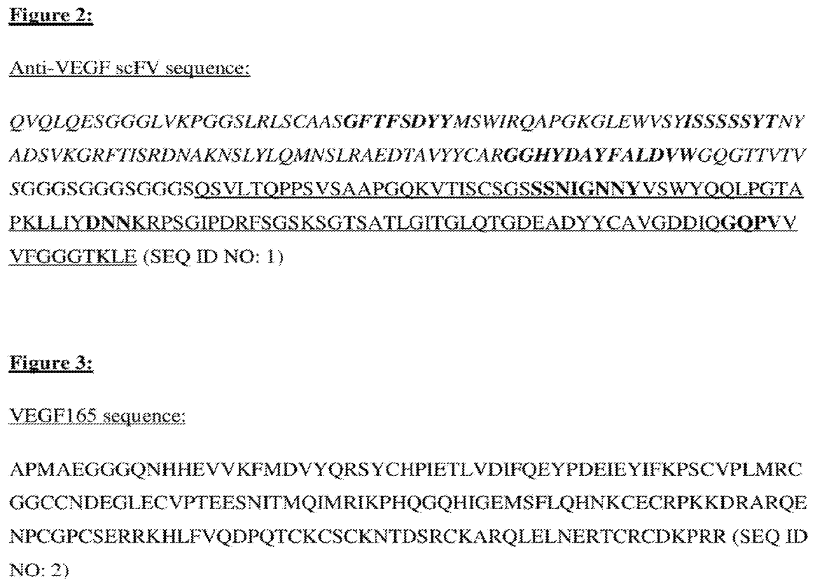

### DESCRIPTION OF THE INVENTION

This invention relates in one aspect to antibodies or antigen-binding fragments thereof that selectively bind human and murine targets, methods of treatment comprising administering said antibodies or antigen-binding fragments, and yeast libraries comprising said antibodies or antigen-binding fragments in display or secretable forms. In one aspect, provided herein are antibodies or antigen-binding fragments that are specific for human and murine Vascular endothelial growth factor (VEGF).

In one aspect, the invention provides antibodies or antigen-binding fragments, which comprise a heavy chain variable region and a light chain variable region, and is specific for human and murine Vascular endothelial growth factor (VEGF). In some embodiments, the antibody or antigen-binding fragment is a single chain Fv (scFv). In some embodiments, the scFv has the amino acid sequence set forth in SEQ ID NO: 1. In some embodiments, the heavy chain variable region comprises a CDR1 sequence of residues 26 to33 of SEQ ID NO: 1; a CDR2 sequence of residues 51 to 58 of SEQ ID NO: 1; and a CDR3 sequence of residues 99 to 111 of SEQ ID NO: 1, and the light chain variable region comprising a CDR1 sequence of residues 158 to 165 of SEQ ID NO: 1; a CDR2 sequence of residues 183 to 185 of SEQ ID NO: 1; and a CDR3 sequence of residues 229 to 232 of SEQ ID NO: 1.

In another aspect, the invention provides methods of treating a tumor in a subject comprising the step of contacting the tumor with an antibody or antigen-binding fragment thereof described herein that is operably linked to a biologically active agent, wherein the agent is a toxin, a radioisotope, a nanoparticle or a bio-active peptide.

In another aspect, the invention provides methods of inhibiting angiogenesis in a solid tumor in a subject, the method comprising the step of contacting the solid tumor with an antibody or antigen-binding fragment thereof described herein that is operably linked to a biologically active agent, wherein the agent is a toxin, a radioisotope, a nanoparticle or a bioactive peptide.

In another aspect, the invention provides methods of inhibiting or suppressing a tumor in a subject, comprising the step of administering an effective amount of an antibody or antigen-binding fragment thereof described herein, wherein the antibody or antigen-binding fragment is operably linked to a biologically active agent, wherein the agent is a toxin, a radioisotope, a nanoparticle or a bio-active peptide.

In another aspect, the invention provides methods of delaying progression of a solid tumor in a subject, the method comprising administering to the subject an effective amount of an antibody or antigen-binding fragment thereof described herein, wherein the antibody or antigen-binding fragment is operably linked to a biologically active agent, wherein the agent is a toxin, a radioisotope, a nanoparticle or a bio-active peptide.

In another aspect, the invention provides methods of diagnosing the presence of a tumor or a cancer growth in a subject, the method comprising sampling a tissue sample isolated from the subject with a composition comprising an antibody or antigen-binding fragment thereof described herein, wherein specific binding of the antibody or antigen-binding fragment to the tissue sample is indicative of the presence of a tumor or cancer growth in the subject.

In another aspect, the invention provides methods of imaging a VEGF-containing tumor, said method comprising the step of applying an antibody or antigen-binding fragment thereof described herein that is operably linked to a secondary reagent; wherein the secondary reagent can be visualized once the antibody or antigen-binding fragment has bound its target VEGF. In some embodiments, the secondary reagent is a photoactivatable agent, a fluorophore, a radioisotope, a bioluminescent protein, a bioluminescent peptide, a fluorescent tag, a fluorescent protein, or a fluorescent peptide.

Compositions according to embodiments of the present invention may further comprise one or more proteolytic inhibitors, pharmaceutical carriers, diluents, adjuvants, or a combination thereof.

In some embodiments, the antibody or antigen-binding fragment is operably linked to a biologically active agent. For example, the biologically active agent is a toxin, a radioisotope, a nanoparticle or a bio-active peptide.

The term “epitope” as used herein refers to a region of the antigen that binds to the antibody or antigen-binding fragment. It is the region of an antigen recognized by a first antibody wherein the binding of the first antibody to the region prevents binding of a second antibody or other bivalent molecule to the region. The region encompasses a particular core sequence or sequences selectively recognized by a class of antibodies. In general, epitopes of protein antigens are comprised by local surface structures or a solvent accessible surface area (SASA) of the proteins 3-dimensional structure that can be formed by contiguous or noncontiguous amino acid sequences.

As used herein, the terms “selectively recognizes”, “selectively bind” or “selectively recognized” mean that binding of the antibody, antigen-binding fragment or other bivalent molecule to an epitope is at least 2-fold greater, preferably 2-5 fold greater, and most preferably more than 5-fold greater than the binding of the molecule to an unrelated epitope or than the binding of an antibody, antigen-binding fragment or other bivalent molecule to the epitope, as determined by techniques known in the art and described herein, such as, for example, ELISA or cold displacement assays.

As used herein, the term “antibody” refers to the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1, Cγ2, and Cγ3. In each pair, the light and heavy chain variable regions (V_{L} and V_{H}) are together responsible for binding to an antigen, and the constant regions (C_{L}, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains V_{H}, Cγ2, and Cγ3. By “immunoglobulin (Ig)” herein is meant a protein consisting of one or more polypeptides substantially encoded by immunoglobulin genes. Immunoglobulins include but are not limited to antibodies Immunoglobulins may have a number of structural forms, including but not limited to full-length antibodies, antibody fragments, and individual immunoglobulin domains including but not limited to V_{H}, Cγ1, Cγ2, Cγ3, V_{L}, and C_{L}.

Depending on the amino acid sequence of the constant domain of their heavy chains, intact antibodies can be assigned to different “classes”. There are five-major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

In some embodiments, the anti-Vascular endothelial growth factor (VEGF) antibody dimer or an isolated anti-VEGF antigen-binding fragment dimer comprises a hinge. In one embodiment, the hinge is an IgG hinge. In some embodiments, the hinge region is from another class of antibodies, as described hereinabove. Thus, in one embodiment, the hinge region is from an IgA. In another embodiment, the hinge region is from an IgD. In another embodiment, the hinge region is from an IgE. In another embodiment, the hinge region is from an IgM.

In one embodiment, the term “antibody” or “antigen-binding fragment” respectively refer to intact molecules as well as functional fragments thereof, such as Fab, a scFv-Fc bivalent molecule, F(ab′)₂, and Fv that are capable of specifically interacting with a desired target. In some embodiments, the antigen-binding fragments comprise:

(1) Fab, the fragment which contains a monovalent antigen-binding fragment of an antibody molecule, which can be produced by digestion of whole antibody with the enzyme papain to yield an intact light chain and a portion of one heavy chain;

(2) Fab′, the fragment of an antibody molecule that can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)2 is a dimer of two Fab′ fragments held together by two disulfide bonds;

(4) Fv, a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

(6) scFv-Fc, is produced in one embodiment, by fusing single-chain Fv (scFv) with a hinge region from an immunoglobulin (Ig) such as an IgG, and Fc regions.

In one embodiment, the IgG is a human IgG. In another embodiment, the IgG is murine. In another embodiment, the IgG is from a non-human primate. In another embodiment, the IgG is porcine, bovine, ovine, equine, caprine, hircine, feline, canine, or avian.

In some embodiments, the antibody provided herein is a monoclonal antibody. In some embodiments, the antigen-binding fragment provided herein is a single chain Fv (scFv), a diabody, a tandem scFv, a scFv-Fc bivalent molecule, an Fab, Fab′, Fv, or F(ab′)₂.

As used herein, the terms “bivalent molecule” or “BY” refer to a molecule capable of binding to two separate targets at the same time. The bivalent molecule is not limited to having two and only two binding domains and can be a polyvalent molecule or a molecule comprised of linked monovalent molecules. The binding domains of the bivalent molecule can selectively recognize the same epitope or different epitopes located on the same target or located on a target that originates from different species. The binding domains can be linked in any of a number of ways including, but not limited to, disulfide bonds, peptide bridging, amide bonds, and other natural or synthetic linkages known in the art (Spatola et al., “Chemistry and Biochemistry of Amino Acids, Peptides and Proteins,” B. Weinstein, eds., Marcel Dekker, New York, p. 267 (1983) (general review); Morley, J. S., “Trends Pharm Sci” (1980) pp. 463-468 (general review); Hudson et al., Int J Pept Prot Res (1979) 14, 177-185; Spatola et al., Life Sci (1986) 38, 1243-1249; Hann, M. M., J Chem Soc Perkin Trans I (1982) 307-314; Almquist et al., J Med Chem (1980) 23, 1392-1398; Jennings-White et al., Tetrahedron Lett (1982) 23, 2533; Szelke et al., European Application EP 45665; Chemical Abstracts 97, 39405 (1982); Holladay, et al., Tetrahedron Lett (1983) 24, 4401-4404; and Hruby, V. J., Life Sci (1982) 31, 189-199).

As used herein, the terms “binds” or “binding” or grammatical equivalents, refer to compositions having affinity for each other. “Specific binding” is where the binding is selective between two molecules. A particular example of specific binding is that which occurs between an antibody and an antigen. Typically, specific binding can be distinguished from non-specific when the dissociation constant (K_{D}) is less than about 1×10⁻⁵ M or less than about 1×10⁻⁶M or 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

In one embodiment, the antibody or antigen-binding fragment binds its target with a K_{D} within the 0.1 nM range. In one embodiment, the antibody or antigen-binding fragment binds its target with a K_{D} of 0.1-2 nM. In another embodiment, the antibody or antigen-binding fragment binds its target with a K_{D} of 0.1-1 nM. In another embodiment, the antibody or antigen-binding fragment binds its target with a K_{D} of 0.05-1 nM. In another embodiment, the antibody or antigen-binding fragment binds its target with a K_{D} of 0.1-0.5 nM. In another embodiment, the antibody or antigen-binding fragment binds its target with a K_{D} of 0.1-0.2 nM.

In one embodiment, the antibody or antigen-binding fragment binds its target with a K_{D} within the 1 nM range. In one embodiment, the antibody or antigen-binding fragment binds its target with a K_{D} of 1-20 nM. In another embodiment, the antibody or antigen-binding fragment binds its target with a K_{D} of 1-10 nM. In another embodiment, the antibody or antigen-binding fragment binds its target with a K_{D} of 0.5-10 nM. In another embodiment, the antibody or antigen-binding fragment binds its target with a K_{D} of 1-5 nM. In another embodiment, the antibody or antigen-binding fragment binds its target with a K_{D} of 1-2 nM.

In one embodiment, the antibody or antigen-binding fragment binds its target with a K_{D} within the 10 nM range. In one embodiment, the antibody or antigen-binding fragment binds its target with a K_{D} of 10-200 nM. In another embodiment, the antibody or antigen-binding fragment binds its target with a K_{D} of 10-100 nM. In another embodiment, the antibody or antigen-binding fragment binds its target with a K_{D} of 5-100 nM. In another embodiment, the antibody or antigen-binding fragment binds its target with a K_{D} of 10-50 nM. In another embodiment, the antibody or antigen-binding fragment binds its target with a K_{D} of 10-20 nM.

In some embodiments, the dissociation constant of an antibody or antigen-binding fragment according to the present invention with its target is at least 5 times lower than the dissociation constant of the corresponding monomer with said target.

In some embodiments, the antibody or antigen-binding fragment thereof provided herein comprises a modification. In another embodiment, the modification minimizes conformational changes during the shift from displayed to secreted forms of the antibody or antigen-binding fragment. It is to be understood by a skilled artisan that the modification can be a modification known in the art to impart a functional property that would not otherwise be present if it were not for the presence of the modification. Encompassed are antibodies which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4, acetylation, formylation, oxidation, reduction, metabolic synthesis in the presence of tunicamycin, etc.

In some embodiments, the modification is one as further defined herein below. In some embodiments, the modification is a N-terminus modification. In some embodiments, the modification is a C-terminal modification. In some embodiments, the modification is an N-terminus biotinylation. In some embodiments, the modification is an C-terminus biotinylation. In some embodiments, the secretable form of the antibody or antigen-binding fragment comprises an N-terminal modification that allows binding to an Immunoglobulin (Ig) hinge region. some embodiments, the Ig hinge region is from but is not limited to, an IgA hinge region. In some embodiments, the secretable form of the antibody or antigen-binding fragment comprises an N-terminal modification that allows binding to an enzymatically biotinylatable site. In some embodiments, the secretable form of the antibody or antigen-binding fragment comprises an C-terminal modification that allows binding to an enzymatically biotinylatable site. In some embodiments, biotinylation of said site functionilizes the site to bind to any surface coated with streptavidin, avidin, avidin-derived moieties, or a secondary reagent.

In some embodiments, the secondary reagent is a protein, a peptide, a carbohydrate, or a glycoprotein.

In some embodiments, the antibody or antigen-binding fragment of the present invention binds to cell lines transduced with human or murine VEGF, cells that express high and moderate levels of endogenous human or murine VEGF, or a combination thereof. In some embodiments, the antibody or antigen-binding fragment of the present invention binds strongly to a cell expressing VEGF. In some embodiments, “strong” binding refers to high affinity binding, binding with a low dissociation rate, or a combination thereof.

In some embodiments, an N-terminal modification of the antibody or antigen-binding fragment provided herein allows fusion of the antibody or antigen-binding fragment with a glycoprotein on the surface of a yeast cell. In some embodiments, the glycoprotein is a protein involved in yeast mating. In some embodiments, the glycoprotein is one involved in lig- and/receptor interactions. In some embodiments, the glycoprotein includes but is not limited to an Aga2. In some embodiments, the antibodies or antigen-binding fragments from the yeast-display library are not biotinylated. In another embodiment, antibodies or antigen-binding fragments from the yeast-display are attached to a yeast surface via a glycoprotein. In yet another embodiment, the glycoprotein is an Aga2 or any glycoprotein known in the art to be useful for binding said antibodies or antigen-binding fragments to a yeast surface.

As used herein, an “isolated peptide” or a “polypeptide” refers to an antibody or antigen-binding fragment as further provided herein. When in reference to any polypeptide of this invention, the term is meant to include native polypeptides (either degradation products, synthetically synthesized peptides or recombinant peptides) and peptidomimetics (typically, synthetically synthesized peptides), such as peptoids and semipeptoids which are peptide analogs, which may have, for example, modifications rendering the polypeptides more stable while in a body or more capable of penetrating into cells. Such modifications include, but are not limited to, N terminal, C terminal or peptide bond modification, including, but not limited to, backbone modifications, and residue modification, each of which represents an additional embodiment of the invention. Methods for preparing peptidomimetic compounds are well known in the art and are specified, for example, in Quantitative Drug Design, C. A. Ramsden Gd., Chapter 17.2, F. Choplin Pergamon Press (1992). In one embodiment, a polypeptide is a full length protein or a variant of a known protein.

Additional post-translational modifications encompassed by the invention include for example, but are not limited to, N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of procaryotic host cell expression.

In some embodiments, the polypeptide comprises an amino acid substitution. In some embodiments, the amino acid substitution is conservative. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). In other embodiments, the amino acid substitution is not a conservative one and that results in enhanced activity of the mutated polypeptide compared to the native polypeptide.

The antibodies or antigen-binding fragments of this invention can be produced by any synthetic or recombinant process such as is well known in the art. The antibodies or antigen-binding fragments of the invention can further be modified to alter biophysical or biological properties by means of techniques known in the art. For example, the polypeptide can be modified to increase its stability against proteases, or to modify its lipophilicity, solubility, or binding affinity to its native receptor.

In some embodiments, antibody fragments may be prepared by proteolytic hydrolysis of the antibody or by expression in E. coli or mammalian cells (e.g. Chinese hamster ovary cell culture or other protein expression systems) of DNA encoding the fragment. Antibody fragments can, in some embodiments, be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R., Biochem. J., 73: 119-126, 1959. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

A “variant” of a polypeptide, antibody, or protein of the present invention as used herein, refers to an amino acid sequence that is altered with respect to the referenced polypeptide, antibody, or protein by one or more amino acids. In the present invention, a variant of a polypeptide retains the antibody-binding property of the referenced protein. As used herein, a “variant” encompasses an antigen-binding fragment of the present invention. Furthermore, a variant encompasses a variant of the antigen-binding fragment that retains specificity for a target or marker. The variant may have “conservative” changes, wherein a substituted amino acid has similar structural or chemical properties (e.g., replacement of leucine with isoleucine). The variants may have conservative amino acid substitutions at one or more predicted non-essential amino acid residues. For example, a “conservative amino acid substitution” may include one in which the amino acid residue is replaced with an amino acid residue having a side chain with a similar charge, whereas, an amino acid residue replaced with an amino acid residue having a side chain with a different charge is a “non-conservative substitution.” Families of amino acid residues having side chains with similar charges have been defined in the art, These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein may routinely be expressed and the functional and/or biological activity of the encoded protein, can be determined using techniques described herein or by routinely modifying techniques known in the art. Analogous minor variations may also include amino acid deletions or insertions, or both. Guidance in determining which amino acid residues may be substituted, inserted, or deleted without abolishing immunological reactivity may be found using computer programs well known in the art, for example, DNASTAR software.

In some embodiments, the antibody or antigen-binding fragment provided herein has a mutation in the heavy chain (VH). In some embodiments, that mutation is a conservative mutation. In other embodiments, that mutation is a non-conservative mutation. In some embodiments, the antibody or antigen-binding fragment provided herein has a mutation in the light chain (VL). In some embodiments, that mutation is a conservative mutation. In other embodiments, that mutation is a non-conservative mutation. In some embodiments, the antibody or antigen-binding fragment provided herein comprises a single mutation. In other embodiments, the antibody or antigen-binding fragment provided herein comprises a combination of mutations.

As used herein, the terms “framework region” or “FR” are those variable domain residues other than the hypervariable region residues. The framework regions have been precisely defined. See, e.g., Kabat, E. A. et al., Sequences of Proteins of Immunological Interest, US Dept. Health and Human Services, National Institutes of Health, USA (5th ed. 1991). Each variable domain typically has four FRs identified as FR1, FR2, FR3 and FR4. As used herein, “FR” also refers to an antibody variable region comprising amino acid residues abutting or proximal to, but outside of the CDR regions i.e. regions which directly interact with the antigen, acting as the recognition element of the antibody molecule within the variable region of an antibody. As used herein, the term “framework region” is intended to mean each domain of the framework that is separated by the CDRs. In some embodiments, the sequences of the framework regions of different light or heavy chains are relatively conserved within a species. The combined heavy and light chain framework regions of an antibody serve to position and align the CDRs for proper binding to the antigen.

The terms “CDR” or “complementarity determining region” refer to amino acid residues comprising non-contiguous antigen combining sites found within the variable region of both heavy and light chain polypeptides. As used herein, the term “CDR” will comprise regions as described by Kabat et al., J. Biol. Chem. 252, 6609-6616 (1977) and Kabat et al., Sequences of protein of immunological interest. (1991), and Chothia and Lesk, J. Mol. Biol. 196:901-917 (1987) and MacCallum et al., J. Mol. Biol. 262:732-745 (1996). The amino acids of the CDRs of the variable domains were initially defined by Kabat, based on sequence variability, to consist of amino acid residues 31-35B (H1), 50-65 (H2), and 95-102 (H3) in the human heavy chain variable domain (VH) and amino acid residues 24-34 (L1), 50-56 (L2), and 89-97 (L3) in the human light chain variable domain (VL), using Kabat's numbering system for amino acid residues of an antibody. See Kabat et al., sequences of proteins of immunological interest, US Dept. Health and Human Services, NIH, USA (5th ed. 1991). Chothia and Lesk, J. Mol. Biol. 196:901-917 (1987) presented another definition of the CDRs based on residues that included in the three-dimensional structural loops of the variable domain regions, which were found to be important in antigen binding activity. Chothia et al. defined the CDRs as consisting of amino acid residues 26-32 (H1), 52-56 (H2), and 95-102 (H3) in the human heavy chain variable domain (VH), and amino acid residues 24-34 (L1), 50-56 (L2), and 89-97 (L3) in the human light chain variable domain (VL). Combining the CDR definitions of Kabat and Chothia, the CDRs consist of amino acid residues 26-35B (H1), 50-65 (H2), and 95-102 (H3) in human VH and amino acid residues 24-34 (L1), 50-56 (L2), and 89-97 (L3) in human VL, based on Kabat's numbering system.

As used herein, a “variable region” when used in reference to an antibody or a heavy or light chain thereof is intended to mean the amino terminal portion of an antibody which confers antigen binding onto the molecule and which is not the constant region. The term is intended to include functional fragments thereof which maintain some of all of the binding function of the whole variable region. Therefore, the term “heteromeric variable region binding fragments” is intended to mean at least one heavy chain variable region and at least one light chain variable regions or functional fragments thereof assembled into a heteromeric complex. Heteromeric variable region binding fragments include, for example, functional fragments such as Fab, F(ab)₂, Fv, single chain Fv (scFv) and the like. Such functional fragments are well known to those skilled in the art. Accordingly, the use of these terms in describing functional fragments of a heteromeric variable region is intended to correspond to the definitions well known to those skilled in the art. Such terms are described in, for example, Harlow and Lane, Antibodies: A Laboratory Manual, Cold Spring Harbor Laboratory, New York (1989); Molec. Biology and Biotechnology: A Comprehensive Desk Reference (Myers, R. A. (ed.), New York: VCH Publisher, Inc.); Huston et al., Cell Biophysics, 22:189-224 (1993); Plückthun and Skerra, Meth. Enzymol., 178:497-515 (1989) and in Day, E. D., Advanced Immunochemistry, Second Ed., Wiley-Liss, Inc., New York, N.Y. (1990).

In some embodiments, a polypeptide of the invention is an isoform of the isolated polypeptide. As used herein, “isoform” refers to a version of a molecule, for example, a protein or polypeptide of the present invention, with only slight differences to another isoform of the same protein or polypeptide. For example, isoforms may be produced from different but related genes, or may arise from the same gene by alternative splicing. Alternatively, isoforms are caused by single nucleotide polymorphisms.

In some embodiments, the isolated polypeptide of this invention is a fragment of the native protein. As used herein, “fragment” refers to a protein or polypeptide that is shorter or comprises fewer amino acids than the full length protein or polypeptide. “Fragment” also refers to a nucleic acid that is shorter or comprises fewer nucleotides than the full length nucleic acid. In some embodiments, the fragment is an N-terminal fragment. In some embodiments, the fragment is a C-terminal fragment. In some embodiments, the fragment is an intrasequential section of the protein, peptide, or nucleic acid. In some embodiments, the fragment is a functional intrasequential section of the protein, peptide or nucleic acid. In some embodiments, the fragment is a functional intrasequential section within the protein, peptide or nucleic acid. In some embodiments, the fragment is an N-terminal functional fragment. In some embodiments, the fragment is a C-terminal functional fragment.

As used herein, the term “functional fragment” refers to a fragment that maintains a certain degree of biological activity as compared to the wild type despite it being a modified version of the native or wild type antibody or polypeptide. This degree of activity could range from moderate to high as compared to the wild type, where the “activity” refers to its natural biophysical or biochemical characteristics, e.g. binding ability, affinity, half-life, stability, etc.

In some embodiments, an isolated polypeptide of this invention comprises a derivative of a polypeptide of this invention. “Derivative” is to be understood as referring to less than the full-length portion of the native sequence of the protein in question. A “derivative” may further comprise (at its termini and/or within said sequence itself) non-native sequences, i.e. sequences which do not form part of the native protein in question. The term “derivative” also includes within its scope molecular species produced by conjugating chemical groups to the amino residue side chains of the native proteins or fragments thereof, wherein said chemical groups do not form part of the naturally-occurring amino acid residues present in said native proteins.

In some embodiments, the invention provides polynucleotides comprising, or alternatively consisting of, a nucleotide sequence encoding an antibody of the invention (including molecules comprising, or alternatively consisting of, antibody fragments or variants thereof). The invention also encompasses polynucleotides that hybridize under high stringency, or alternatively, under intermediate or lower stringency hybridization conditions to polynucleotides complementary to nucleic acids having a polynucleotide sequence that encodes an antibody according to embodiments of the invention or a fragment or variant thereof.

The polynucleotides are obtained, and the nucleotide sequence of the polynucleotides determined, by any method known in the art. In some cases, a polynucleotide encoding an antibody (including molecules comprising, or alternatively consisting of, antibody fragments or variants thereof) are generated from nucleic acid from a suitable source. If a clone containing a nucleic acid encoding a particular antibody is not available, but the sequence of the antibody molecule is known, a nucleic acid encoding it may be chemically synthesized or obtained from a suitable source (e.g., an antibody cDNA library, or a cDNA library generated from, or nucleic acid, preferably poly A+RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art

Methods of making antibodies and antibody fragments are known in the art. (See for example, Harlow and Lane, Antibodies: A Laboratory Manual, Cold Spring Harbor Laboratory, New York, 1988, incorporated herein by reference).

Antibodies can be produced by the immunization of various animals, including mice, rats, rabbits, goats, primates, humans and chickens with a target antigen such as VEGF or peptide fragments of VEGF containing an anti-VEGF epitope of the present invention. In some embodiments, the antibody or antigen-binding fragment of the present invention can be purified by methods known in the art, for example, gel filtration, ion exchange, affinity chromatography, etc. Affinity chromatography or any of a number of other techniques known in the art can be used to isolate polyclonal or monoclonal antibodies from serum, as-cites fluid, or hybridoma supernatants.

“Purified” means that the antibody or antigen-binding fragment is separated from at least some of the proteins normally associated with the antibody or antigen-binding fragment and preferably separated from all cellular materials other than proteins.

As used herein, the term “nucleic acid” refers to polynucleotide or to oligonucleotides such as deoxyribonucleic acid (DNA), and, where appropriate, ribonucleic acid (RNA) or mimetic thereof. The term should also be understood to include, as equivalents, analogs of either RNA or DNA made from nucleotide analogs, and, as applicable to the embodiment being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

As will be appreciated by one skilled in the art, a fragment or derivative of a nucleic acid sequence or gene that encodes for a protein or peptide can still function in the same manner as the entire, wild type gene or sequence. Likewise, forms of nucleic acid sequences can have variations as compared to wild type sequences, nevertheless encoding a protein or peptide, or fragments thereof, retaining wild type function exhibiting the same biological effect, despite these variations.

The nucleic acids of the present invention can be produced by any synthetic or recombinant process such as is well known in the art. Nucleic acids according to the invention can further be modified to alter biophysical or biological properties by means of techniques known in the art. For example, the nucleic acid can be modified to increase its stability against nucleases (e.g., “end-capping”), or to modify its lipophilicity, solubility, or binding affinity to complementary sequences.

Methods for modifying nucleic acids to achieve specific purposes are disclosed in the art, for example, in Sambrook et al. (1989). Moreover, the nucleic acid sequences of the invention can include one or more portions of nucleotide sequence that are non-coding for the protein or polypeptide of interest. The invention further provides DNA sequences which encode proteins or polypeptides similar to those encoded by sequences as described herein, but which differ in terms of their codon sequence due to the degeneracy of the genetic code or allelic variations (naturally-occurring base changes in the species population which may or may not result in an amino acid change), which may encode the proteins of the invention described herein, as well. Variations in the DNA sequences, which are caused by point mutations or by induced modifications (including insertion, deletion, and substitution) to enhance the activity, half-life or production of the polypeptides encoded thereby, are also encompassed in the invention.

DNA encoding the antibodies or antigen-binding fragments provided herein is readily isolated and sequenced using conventional procedures (e.g., by using oligonucleotide probes that are capable of binding specifically to genes encoding the heavy and light chains of the antibodies). Hybridoma cells serve as a source of such DNA. Once isolated, the DNA may be placed into expression vectors, which are then transfected into host cells such as E. coli cells, simian COS cells, Chinese hamster ovary (CHO) cells, yeast cells or myeloma cells that do not otherwise produce immunoglobulin protein, to obtain the synthesis of the antibodies in the recombinant host cells. Recombinant production of antibodies is described in more detail below.

It is to be understood by a skilled artisan that the antibody, antigen-binding fragments, or compositions provided herein can be used in therapeutic or diagnostic procedures.

As used herein, the term “operably linked” refers to the positioning/linking of the two or more molecules or sequences in such a manner as to ensure the proper function or expression of the molecule and sequence.

As used herein, the term “therapeutically effective amount” refers to an amount that provides a therapeutic effect for a given condition and administration regimen.

As used herein, the term “preventing, or treating” refers to any one or more of the following: delaying the onset of symptoms, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, preventing relapse to a disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics. It is understood that “treating” may refer to both therapeutic treatment and prophylactic or preventive measures, wherein the object is to prevent or lessen the targeted pathologic condition or disorder.

As used herein, “symptoms” are manifestation of a disease or pathological condition as described hereinabove.

In some embodiments, the compositions of this invention comprise a polypeptide, antibody, or antigen-binding fragment of this invention, alone or in some embodiments, in combination with a second pharmaceutically active agent. As used herein, the term “pharmaceutically active agent” refers to any medicament which satisfies the indicated purpose. Examples of pharmaceutically active agents include, but are not limited to a decongestant, antibiotic, bronchodilator, anti-inflammatory steroid, leukotriene antagonist or histamine receptor antagonist, and the like.

In yet another aspect, provided herein are methods of delivering a biologically active agent and the antibody or antigen-binding fragment of the present invention for the treatment of a tumor in a subject. In some embodiments, the methods comprise the step of concomitantly but individually administering the biologically active agent and the antibody or antigen-binding fragment. In some embodiments, the methods comprise the step of separately administering the biologically active agent and the antibody or antigen-binding fragment. In some embodiments, the methods comprise the step administering of the biologically active agent and the antibody or antigen-binding fragment in a single formulation. In some embodiments, the methods comprise the step of administering the biologically active agent and the antibody or antigen-binding fragment in separate formulations. In some embodiments, the methods comprise the step of administering the biologically active agent and the antibody or antigen-binding fragment at or around the same time. In some embodiments, the methods comprise the step of administering the biologically active agent and the antibody or antigen-binding fragment at different times, which may entail a separation of one or more hours or one or more days.

In some embodiments, the antibody or antigen-binding fragment provided herein are themselves “biologically active”, meaning they are able to exert the biological action or an enhanced action of their corresponding parental antibodies even after modification, in particular in binding to the target antigen, inhibiting binding of ligands to receptors, further in terms of modulation, in particular inhibition of antigen-mediated signal transduction and prophylaxis or therapy of antigen-mediated diseases. The term “biologically active”, when used in reference to any of the biologically active agents described herein also refers to the agent's ability to modulate the immune response in a manner that can lead to a preventive, diagnostic, or therapeutic effect as will be understood by a skilled artisan. In some embodiments, agents that are used to achieve this biological activity include but are not limited to a cytokine, an enzyme, a chemokine, a radioisotope, an enzymatically active toxin, a therapeutic nano particle or a chemotherapeutic agent, as will be understood by a skilled artisan.

In some embodiments, the polypeptides of antibodies are conjugated or operably linked so as to function in their intended purpose to an enzyme in order to employ Antibody Dependent Enzyme Mediated Prodrug Therapy (ADEPT). ADEPT may be used by conjugating or operably linking an antibody or antigen binding fragment provided herein to a pro-drug-activating enzyme that converts a prodrug (e.g. a peptidyl chemotherapeutic agent) to an active anti-cancer drug. The enzyme component of the immunoconjugate useful for ADEPT includes any enzyme capable of acting on a prodrug in such a way so as to convert it into its more active, cytotoxic form. Enzymes that are useful in the method of this invention include but are not limited to alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuramimidase useful for converting glycosylated prodrugs into free drugs; β-lactamase useful for converting drugs derivatized with a-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Antibodies with enzymatic activity, also known in the art as “abzymes”, can be used to convert the prodrugs into free active drugs (see, for example, Massey, 1987, Nature 328: 457-458). Polypeptide/antibody-abzyme conjugates can be prepared for delivery of the abzyme to a tumor cell population. Other additional modifications of the modified molecules provided herein are contemplated herein. For example, the polypeptide/antibody may be linked to one of a variety of nonproteinaceous polymers, e.g., polyethylene glycol (PEG), polypropylene glycol, polyoxyalkylenes, or copolymers of polyethylene glycol and polypropylene glycol.

In some embodiments, the antibody/polypeptide provided herein is administered with one or more immunomodulatory agents. Such agents may increase or decrease production of one or more cytokines, up- or down-regulate self-antigen presentation, mask MHC antigens, or promote the proliferation, differentiation, migration, or activation state of one or more types of immune cells. Immunomodulatory agents include but are not limited to: non-steroidal anti-inflammatory drugs (NSAIDs) such as aspirin, ibuprofen, celecoxib, diclofenac, etodolac, fenoprofen, indomethacin, ketoralac, oxaprozin, nabumentone, sulindac, tolmentin, rofecoxib, naproxen, ketoprofen, and nabumetone; steroids (e.g. glucocorticoids, dexamethasone, cortisone, hydroxycortisone, methylprednisolone, prednisone, prednisolone, trimcinolone, azulfidineicosanoids such as prostaglandins, thromboxanes, and leukotrienes; as well as topical steroids such as anthralin, calcipotriene, clobetasol, and tazarotene); cytokines such as TGFb, IFNa, IFNb, IFNg, IL-2, IL-4, IL-10; cytokine, chemokine, or receptor antagonists including antibodies, soluble receptors, and receptor-Fc fusions against BAFF, B7, CCR2, CCR5, CD2, CD3, CD4, CD6, CD7, CD8, CD11, CD14, CD15, CD17, CD18, CD20, CD23, CD28, CD40, CD40L, CD44, CD45, CD52, CD64, CD80, CD86, CD147, CD152, complement factors (C5, D) CTLA4, eotaxin, Fas, ICAM, ICOS, IFN-α IFN-β, IFN-γ., IFNAR, IgE, IL-1, IL-2, IL-2R, IL-4, IL-5R, IL-6, IL-8, IL-9 IL-12, IL-13, IL-13R1, IL-15, IL-18R, IL-23, integrins, LFA-1, LFA-3, MHC, selectins, TGF-β, TNF-α, TNF-β, TNF-R1, T-cell receptor, including Enbrel®. (etanercept), Humira®. (adalimumab), and Remicade®. (infliximab); heterologous anti-lymphocyte globulin; other immunomodulatory molecules such as 2-amino-6-aryl-5 substituted pyrimidines, anti-idiotypic antibodies for MHC binding peptides and MHC fragments, azathioprine, brequinar, bromo-cryptine, cyclophosphamide, cyclosporine A, D-penicillamine, deoxyspergualin, FK506, glutaraldehyde, gold, hydroxychloroquine, leflunomide, malononitriloamides (e.g. leflunomide), methotrexate, minocycline, mizoribine, mycophenolate mofetil, rapamycin, and sulfasasazine.

In some embodiments, antibodies of the present invention are administered with a cytokine. By “cytokine” as used herein is meant a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and -II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

A chemotherapeutic or other cytotoxic agent may be administered as a prodrug. The term “prodrug” refers to a precursor or derivative form of a pharmaceutically active substance that is less cytotoxic to tumor cells compared to the parent drug and is capable of being enzymatically activated or converted into the more active parent form. See, for example Wilman, 1986, Biochemical Society Transactions, 615th Meeting Belfast, 14:375-382; and Stella et al., “Prodrugs: A Chemical Approach to Targeted Drug Delivery,” Directed Drug Delivery, Borchardt et al., (ed.): 247-267, Humana Press, 1985. The prodrugs that may find use with the compositions and methods as provided herein include, but are not limited to, phosphate-containing prodrugs, thiophosphate-containing prodrugs, sulfate-containing prodrugs, peptide-containing prodrugs, D-amino acid-modified prodrugs, glycosylated prodrugs, beta-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use with the antibodies/polypeptides of the compositions and methods provided herein include, but are not limited to, any of the aforementioned chemotherapeutic agents.

In some embodiments, any combination of the antibody/polypeptide with the biological active agents specified above, i.e., a cytokine, an enzyme, a chemokine, a radioisotope, an enzymatically active toxin, or a chemotherapeutic agent can be applied. In some embodiments, the antibody/polypeptide can be operably-linked with the biologically active agent and used in the methods described herein or antibody/polypeptide provided herein can merely be used in combination with the biologically active agents, in a manner in which both are administered separately (i.e.—not conjugated) to achieve the desired preventive, diagnostic, or therapeutic effect.

What is claimed is:

1. An antibody or antigen-binding fragment thereof, comprising a heavy chain variable region and a light chain variable region, and specifically binds to a human Vascular endothelial growth factor (VEGF). wherein the heavy chain variable region comprises a CDR1 sequence of residues 26 to33 of SEQ ID NO: 1; a CDR2 sequence of residues 51 to 58 of SEQ ID NO: 1; and a CDR3 sequence of residues 99 to 111 of SEQ ID NO: 1, and the light chain variable region comprising a CDR1 sequence of residues 158 to 165 of SEQ ID NO: 1; a CDR2 sequence of residues 183 to 185 of SEQ ID NO: 1; and a CDR3 sequence of residues 229 to 232 of SEQ ID NO: 1.

2. The antibody or antigen-binding fragment of claim 1, wherein the heavy chain variable region has the amino acid sequence of residues 1 to 120 of SEQ ID NO: 1

3. The antibody or antigen-binding fragment of claim 1, wherein the light chain variable region has the amino acid sequence of residues 133 to 242 of SEQ ID NO: 1.

4. The antibody or antigen-binding fragment of claim 1, wherein the heavy chain variable region and light chain variable region have the amino acid sequences of residues 1 to 120 and 133 to 242 of SEQ ID NO: 1

5. The antibody of claim 1, wherein the antibody or antigen-binding fragment comprises a modification.

6. The antibody or antigen-binding fragment of claim 5, wherein said modification is a N-terminus modification.

7. The antibody or antigen-binding fragment of claim 5, wherein said modification is a C-terminus modification.

8. The antibody or antigen-binding fragment of claim 5, wherein said modification is biotinylation.

9. The antibody or antigen-binding fragment of claim 1, wherein said antigen-binding fragment is a single chain Fv (scFv), a scFv-Fc bivalent molecule, an Fab, Fab′, Fv, or F(ab′)2.

10. The antibody or antigen-binding fragment of claim 9, wherein said antigen-binding fragment is a single chain Fv (scFv).

11. The antibody or antigen-binding fragment of claim 10, wherein the scFv has the amino acid sequence set forth in SEQ ID NO: 1.

12. The antibody or antigen-binding fragment of claim 1, wherein said antibody is a monoclonal antibody.

13. A method of treating a tumor in a subject comprising the step of contacting said tumor with the antibody or antigen-binding fragment thereof according to any one of claims 1-12, wherein the antibody or antigen-binding fragment is operably linked to a biologically active agent, wherein said agent is a toxin, a radioisotope, a nanoparticle or a bio-active peptide. antibody.

14. A method of inhibiting angiogenesis in a solid tumor in a subject, said method comprising the step of contacting said solid tumor with the antibody or antigen-binding fragment thereof according to any one of claims 1-12, wherein the antibody or antigen-binding fragment is operably linked to a biologically active agent, wherein said agent is a toxin, a radioisotope, a nanoparticle or a bio-active peptide.

15. A method of inhibiting or suppressing a tumor in a subject, comprising the step of administering an effective amount of the antibody or antigen-binding fragment thereof according to any one of claims 1-12, wherein the antibody or antigen-binding fragment is operably linked to a biologically active agent, wherein said agent is a toxin, a radioisotope, a nanoparticle or a bio-active peptide.

16. A method of delaying progression of a solid tumor in a subject, said method comprising administering to said subject an effective amount of the antibody or antigen-binding fragment thereof according to any one of claims 1-12, wherein the antibody or antigen-binding fragment is operably linked to a biologically active agent, wherein said agent is a toxin, a radioisotope, a nanoparticle or a bio-active peptide.

17. A method of diagnosing the presence of a tumor or a cancer growth in a subject, said method comprising sampling a tissue sample isolated from said subject with a composition comprising the antibody or antigen-binding fragment thereof according to any one of claims 1-12, wherein the antibody or antigen-binding fragment, wherein specific binding of said antibody or antigen-binding fragment to said tissue sample is indicative of the presence of a tumor or cancer growth in said subject.

18. The method of claim 17, further comprising a secondary reagent that specifically binds to said antibody or antigen-binding fragment but does not antagonize binding of said antibody or antigen-binding fragment to its target.

19. The method of claim 18, wherein said secondary reagent is a photoactivatable agent, a fluorophore, a radioisotope, a bioluminescent protein, a bioluminescent peptide, a fluorescent tag, a fluorescent protein, or a fluorescent peptide.

20. The method of claim 17, wherein said sampling comprises the step of analyzing said sample using a chromogenic immunological assay.

21. The method of claim 17, wherein said sampling comprises the step of analyzing said sample using microscopic imaging.

22. A method of imaging a VEGF-containing tumor, said method comprising the step of applying the antibody or antigen-binding fragment thereof according to any one of claims 1-12, wherein the antibody or antigen-binding fragment is operably linked to a secondary reagent; and wherein said secondary reagent can be visualized once said antibody or antigen-binding fragment has bound its target VEGF.

23. The method of claim 22, wherein said secondary reagent is a photoactivatable agent, a fluorophore, a radioisotope, a bioluminescent protein, a bioluminescent peptide, a fluorescent tag, a fluorescent protein, or a fluorescent peptide.


SEQUENCE LISTING (2 sequences)

SEQ ID NO: 1 - protein, 242 aa, Homo sapiens
QVQLQESGGGLVKPGGSLRLSCAASGFTFSDYYMSWIRQAPGKGLEWVSYISSSSSYTNYADSVKGRFTISRDNAKNSLYLQMNSLRAEDTAVYYCARGGHYDAYFALDVWGQGTTVTVSGGGSGGGSGGGSQSVLTQPPSVSAAPGQKVTISCSGSSSNIGNNYVSWYQQLPGTAPKLLIYDNNKRPSGIPDRFSGSKSGTSATLGITGLQTGDEADYYCAVGDDIQGQPVVVFGGGTKLE

SEQ ID NO: 2 - protein, 165 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR